(19) United States
(12) Patent Application Publication
(10) Pub. No.: US 2025/0152923 A1
(43) Pub. Date: May 15, 2025

(54) VASCULAR STENOSIS TREATMENT APPARATUS

(71) Applicant: SUZHOU VENMED TECHNOLOGY CO., LTD., Suzhou (CN)

(72) Inventors: Xiaoqiang Li, Suzhou (CN); Haiquan Feng, Suzhou (CN); Xiaotian Wang, Suzhou (CN); Yonggang Wang, Suzhou (CN); Shuheng Dong, Suzhou (CN); Jian Shi, Suzhou (CN); Luzi Bao, Suzhou (CN)

(73) Assignee: SUZHOU VENMED TECHNOLOGY CO., LTD., Suzhou, JS (CN)

(21) Appl. No.: 18/725,380

(22) PCT Filed: Nov. 15, 2022
(86) PCT No.: PCT/CN2022/131923

§ 371 Date: Jun. 28, 2024

(30) Foreign Application Priority Data
Dec. 31, 2021 (CN) 202111669879.5

(51) Int. Cl. A61M 25/10

(52) U.S. Cl. CPC A61M 25/104; A61M 2025/105; A61M 2025/1081; A61M 2025/109

(57) ABSTRACT

Embodiments herein are directed to a vascular stenosis treatment apparatus including a treatment assembly having a balloon, the wall of the balloon being provided with a plurality of first holes for liquid to pass through, and the balloon having an unexpanded state and an expanded state; a covering member covering the outside of the balloon and expanding with the expansion of the balloon, the covering member having a plurality of second holes, and when the balloon is in the expanded state, the aperture of the first holes being smaller than the aperture of the second holes. The treatment apparatus can achieve accurate positioning, and the covering member has a limiting effect on a drug solution causing the drug to more uniformly and effectively act on a desired position of a vascular wall, increasing the utilization rate, reducing the occurrence rate of vascular restenosis, and improving the treatment effect.

### CROSS-REFERENCE TO RELATED PATENT APPLICATIONS

This application is a U.S. national phase application of, and claims the benefit of and priority to, International Patent Application No. PCT/CN2022/131923, filed Nov. 15, 2022, which claims the benefit of and priority to Chinese Patent Application CN202111669879.5, filed Dec. 31, 2021, the contents of which are both hereby incorporated by reference in their entirety.

### TECHNICAL FIELD

The present disclosure belongs to the field of medical apparatus and instruments, in particular relates to a vascular stenosis treatment apparatus.

### BACKGROUND

Since the 1970s, percutaneous transluminal balloon angioplasty has been used to treat vascular stenosis caused by atherosclerosis and reconstruct the blood supply of occluded vessels, balloon angioplasty widens the stenosis and occlusion area, and makes blood flow better by inserting a catheter with a dilation balloon into the vascular system, and under external pressure, expanding the balloon at the stenosis site of the vessel, so as to apply radial pressure to the inner wall of the vessel. Although the immediate effect of balloon angioplasty is satisfactory, the excessive expansion of the balloon during catheter dilation can damage blood vessels, or the insufficient performance of the balloon itself during the treatment of severe hard calcification lesions cannot effectively dilate blood vessels, resulting in a high incidence of surgical complications, especially restenosis in balloon angioplasty, which limits its widespread clinical application.

In order to improve the therapeutic effect of balloons, drug coated balloons have been introduced in prior art, however, the drug binding force on the surface of the balloons is weak, therefore, during the process of transporting the balloons to the lesion sites, a large amount of drug has been lost and the amount of drug loss is uncontrollable, resulting in significant fluctuations in the drug concentration received by the blood vessel wall.

Another method for treating vascular stenosis in prior art is endovascular stenting angioplasty, which is to implant a stent at the intravascular stenosis site, and can effectively reduce the rate of endovascular restenosis, but 20-30% of patients still experience endovascular restenosis, which is difficult to treat.

In order to improve the therapeutic effect of endovascular stenting angioplasty, drug coated stents have emerged in prior art, and due to the presence of polymer coatings loaded with drugs on the stents, long-term stimulation is generated between the stents and the inner wall of the vessel, which can easily lead to late thrombosis. In addition, for stenosis lesions with severe calcification, the inability of the stents to fully adhere to the wall results in the inability of the drugs to function, ultimately leading to restenosis within the stents.

Due to the risk of massive bleeding, pharmacological thrombolysis is mostly used in clinical emergency treatment, and thrombolytic drugs cannot be continuously injected for a long time (generally not exceeding 3-5 days), otherwise it is easy to cause adverse reactions such as massive bleeding. Mechanical thrombectomy pulls the thrombus into the proximal catheter by proximal vacuum aspiration or distal contact with the thrombus to remove the thrombus and restore blood perfusion. However, due to the difficulty in breaking old thrombi, they often cannot smoothly enter the aspiration catheter or be captured by mechanical thrombectomy apparatuses such as the catheter thrombectomy mesh, which may lead to adverse consequences such as prolonged thrombectomy time and excessive blood loss in patients.

### SUMMARY

The present disclosure is aimed to provided a vascular stenosis treatment apparatus with a different structure.

To solve the above technical problems, the present disclosure employs the following technical solution:

A vascular stenosis treatment apparatus, including a treatment assembly, the treatment assembly having:
a balloon, the wall of the balloon being provided with a plurality of first holes for liquid to pass through, and the balloon having an unexpanded state and an expanded state;
a covering member, the covering member covering the outside of the balloon and expanding with the expansion of the balloon, the covering member having a plurality of second holes, and when the balloon is in the expanded state, the aperture of the first holes being smaller than the aperture of the second holes.

Specifically, when the balloon transitions from the unexpanded state to the expanded state, both the first holes on the balloon and the second holes on the covering member become larger, and when the balloon is in the expanded state, the degree of enlargement of the second holes is greater than that of the first holes.

Specifically, the coverage rate of the peripheral surface of the balloon by the covering member in the unexpanded state is greater than that of the peripheral surface of the balloon by the covering member in the expanded state.

The coverage rate in the unexpanded state refers to, when both the covering member and the balloon are in the unexpanded state, and the roughly cylindrical peripheral surface of the balloon is 100%, the percentage of the total projection area of the covering member on the cylinder to the peripheral surface of the balloon.

The coverage rate in the expanded state refers to the percentage of the total projection area of the covering member on the peripheral surface of the balloon to the peripheral surface of the balloon when both the covering member and the balloon are in the expanded state.

According to some embodiments, in the expanded state, the position of the plurality of first holes corresponds to the position of the plurality of second holes, such that a plurality of relatively independent drug delivery spaces are formed between the balloon and the covering member.

According to some embodiments, in the unexpanded state, the position of the first holes corresponds to the position of the second holes.

According to some embodiments, one second hole is provided corresponding to one or more first holes.

Further, when one second hole is provided corresponding to one first hole, the axis line of the first hole coincides with the axis line of the second hole; when one second hole is provided corresponding to two or more first holes, the two or more first holes are uniformly distributed within the range of the second hole.

Further, one second hole is provided corresponding to 2˜5 first holes.

Specifically, the number and aperture of the first holes are provided so that, in the expanded state, the drug solution can be sprayed out from the first holes, and in the unexpanded state and during the process of moving from the unexpanded state to the expanded state, the drug solution remains inside the balloon or only a small amount of drug solution seeps out from the first holes.

According to some embodiments, in the expanded state, the cross-sectional area of the second holes is 10˜50 mm².

According to some embodiments, in the expanded state, the shape of the second holes is rhombus.

According to some embodiments, in the expanded state, the aperture of the first holes is 10˜80 μm.

According to some embodiments, the distal end of the covering member is fixedly connected to the distal end of the balloon, and the proximal end of the covering member is fixedly connected to the proximal end of the balloon.

According to some embodiments, the covering member is provided that part or all of the covering member moves relative to the outer surface of the balloon to generate a cutting action when the balloon is expanded.

Further, when the balloon transitions from the unexpanded state to the expanded state, the contact area between part or all of the covering member and the peripheral surface of the balloon is changed to realize the cutting of an intravascular lesion site by the covering member.

According to some embodiments, the covering member includes a plurality of covering elements and a plurality of connection points;
when the covering member is in the unexpanded state, each of the covering elements has a length extending along the axial direction of the covering member, and the plurality of covering elements is distributed along a circumferential direction;
two adjacent covering elements are fixedly connected through the plurality of connection points, and two adjacent connection points on the same covering element are respectively located on two opposite sides of the covering element and are staggered with each other;
when the covering member transitions from the unexpanded state to the expanded state, the covering elements are flipped, and two adjacent connection points connected to the same covering element move respectively towards directions close to the balloon and away from the balloon.

More further, the plurality of connection points are respectively located in a plurality of planes parallel to each other, the planes are perpendicular to the axis line of the covering member, and the distances between two adjacent planes are equal.

More further, when the covering member is in the unexpanded state, the width of the covering elements is greater than the thickness of the covering elements.

More further, when the covering member is in the unexpanded state, the width of the covering elements is 2˜10 times the thickness of the covering elements.

More further, the connection points have a length of 0.5˜3 mm.

More further, the connection points have a thickness of 0.02˜0.2 mm.

More further, the connection points have a width of 0.1˜0.5 mm.

More further, the thickness of the connection points is equal to the thickness of the covering elements.

More further, the plurality of covering elements and the plurality of connection points together form the plurality of second holes, and in the expanded state, the covering elements enclose outside the first holes to form a plurality of independent drug delivery spaces.

According to some embodiments, the covering member is formed by cutting a cylindrical tube.

According to some embodiments, the material of the covering member is metal, including but not limited to the group consisting of memory alloy, stainless steel, titanium alloy, nickel alloy, cobalt-chromium alloy, and combinations thereof.

According to some embodiments, when the covering member is in the expanded state, the second holes are rhombic, and in the same rhombus, the connection points at two opposite vertexes of this rhombus are located on the inner side of the covering member, and the connection points at the other two opposite vertexes of this rhombus are located on the outer side of the covering member.

According to some embodiments, the covering member further includes a first connecting member located at the distal end of the covering member and a second connecting member located at the proximal end of the covering member, the first connecting member is fixedly connected to the distal end of the balloon and the distal ends of the covering elements, respectively, and the second connecting member is fixedly connected to the proximal end of the balloon and the proximal ends of the covering elements, respectively.

According to some embodiments, the covering member further includes a first connecting member located at the distal end of the covering member, a second connecting member located at the proximal end of the covering member, a first extension member fixedly connected to the proximal end of the first connecting member and the distal ends of the covering elements and capable of being elongated or shortened, and a second extension member fixedly connected to the distal end of the second connecting member and the proximal ends of the covering elements and capable of being elongated or shortened, the first connecting member is fixedly connected to the distal end of the balloon, and the second connecting member is fixedly connected to the proximal end of the balloon; the length of the first extension member when the balloon is in the unexpanded state is less than that of the first extension member when the balloon is in the expanded state, and the length of the second extension member when the balloon is in the unexpanded state is less than that of the second extension member when the balloon is in the expanded state.

More further, the balloon includes a first head portion at the distal end, a second head portion at the proximal end, and a balloon body fixedly connected to the first head portion and the second head portion, the first connecting member is fixedly connected to the first head portion, the second connecting member is fixedly connected to the second head portion, and the covering elements extend on the peripheral surface of the entire balloon.

More further, the first connecting member and the second connecting member are respectively circular rings, or are respectively closed rings having peaks and valleys regularly arranged along the circumferential direction with the axis line of the covering member as the axis.

More further, in the unexpanded state, the first extension member and the second extension member are respectively a plurality of wavy lines uniformly distributed along the circumferential direction with the axis line of the covering member as the axis.

More further, in the expanded state, the connections between the first extension member and the covering elements and the connections between the second extension member and the covering elements are located in the positions where the diameter of the balloon becomes to increase when it transitions from the unexpanded state to the expanded state.

More further, the connection between the first connecting member and the first extension member and the connection between the second connecting member and the second extension member are located in the positions where the diameter of the balloon becomes to increase when it transitions from the unexpanded state to the expanded state.

Further, during or after the covering member carries out the cutting, the drug solution is sprayed out from the first holes.

According to some embodiments, in the expanded state and/or during the process of moving from the unexpanded state to the expanded state, the balloon and the covering member are attached to each other. That is, the covering member cannot be self-expanded, and can only be expanded under the force of balloon expansion.

Further, the vascular stenosis treatment apparatus further includes a first catheter partially located within the balloon and connected to the distal end of the balloon in a sealed manner or integrally formed with the distal end of the balloon, a second catheter sleeved outside the first catheter and connected to the proximal end of the balloon in a sealed manner or integrally formed with the proximal end of the balloon, and a catheter seat respectively connected to the proximal end of the first catheter and the proximal end of the second catheter, forming a drug delivery channel between the first catheter and the second catheter.

The present disclosure has the following advantages over the prior art:

The vascular stenosis treatment apparatus of the present disclosure can achieve accurate positioning, and the covering member has a limiting effect on a drug solution, so that the drug can more uniformly and effectively act on a desired position of a vascular wall, thus further increasing the utilization rate of the drug, reducing the occurrence rate of vascular restenosis, and improving the treatment effect.

### BRIEF DESCRIPTION OF THE DRAWINGS

FIG. 1 is a front view of a concrete implementation of a vascular stenosis treatment apparatus in the unexpanded state;

FIG. 2 is a partially sectional view of the concrete implementation of the vascular stenosis treatment apparatus in the unexpanded state;

FIG. 3 is a front view of the concrete implementation of the vascular stenosis treatment apparatus in the expanded state;

FIG. 4 is a partially sectional view of the concrete implementation of the vascular stenosis treatment apparatus in the expanded state;

FIG. 5 is an enlarged view of the distal portion of FIG. 2;

FIG. 6 is an enlarged view of the distal portion of FIG. 4;

FIG. 7 is an enlarged view of Part A in FIG. 6;

FIG. 8 is a front view of an implementation of a covering member in the unexpanded state;

FIG. 9 is a front view of the implementation of the covering member in the expanded state;

FIG. 10 is a sectional view along B-B in FIG. 9;

FIG. 11 is a front view of another implementation of the relative position of the first holes and the second holes in the expanded state;

FIG. 12 is a front view of still another implementation of the relative position of the first holes and the second holes in the expanded state;

FIG. 13 is a front view of another implementation of a covering member in the unexpanded state;

FIG. 14 is a front view of another implementation of a covering member in the expanded state;

FIG. 15 is a front view of still another implementation of a covering member in the unexpanded state; and

FIG. 16 is a front view of still another implementation of a covering member in the expanded state.

### DETAILED DESCRIPTION

In order to make the above purposes, features and advantages of this disclosure more clearly understood, specific implementations of this disclosure will be described in detail in conjunction with the accompanying drawings. In the following description, numerous specific details are set forth so as to provide a thorough understanding of this disclosure. However, this disclosure can be implemented in many other ways different from those described herein, and those skilled in the art can make similar improvements without departing from the connotation of this disclosure, therefore, this disclosure is not limited by the specific embodiments disclosed below. The technical features involved in the different implementations of this disclosure can be combined with each other as long as they do not conflict with each other.

In the description of this disclosure, it is necessary to understand that the distal end and the proximal end are defined from the operator's point of view, the distal end refers to the end away from the operator, and the proximal end refers to the end near the operator.

The orientation or positional relationship indicated by the terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial direction”, “radial direction”, “circumferential direction”, and the like is based on the orientation or positional relationship shown in the accompanying drawings, is only for the convenience of describing this application and simplifying the description, rather than indicating or implying that the indicated device or element must have a particular orientation, be constructed and operate in a particular orientation, and therefore should not be construed as limiting this disclosure.

Furthermore, the terms “first” and “second” are used for descriptive purposes only and should not be construed to indicate or imply relative importance or implicitly indicate the number of technical features indicated. Therefore, the features limited to “first” and “second” can explicitly or implicitly include at least one of said features. In the description of this disclosure, “a plurality of” means at least two, such as two, three, etc., unless otherwise expressly and specifically defined.

In this disclosure, unless otherwise expressly specified and limited, the terms “mount”, “communicate”, “connect”, “fix” and other terms should be understood in a broad sense, for example, it may be fixedly connected or detachably connected, or integrated; it can be directly connected or indirectly connected through an intermediate medium, or it can be the internal communication of two elements or the interaction relationship between two elements, unless otherwise expressly limited. For those of ordinary skill in the art, the specific meanings of the above terms in this application can be understood according to specific situations.

In this disclosure, unless otherwise expressly specified and limited, a first feature being “on” or “under” a second feature may mean that the first feature is in direct contact with the second feature, or the first feature is in indirect contact with the second feature through an intermediate medium. Also, the first feature being “on”, “above”, or “over” the second feature may mean that the first feature is directly above or obliquely above the second feature, or simply means that the first feature is level higher than the second feature. The first feature being “under”, “below” or “underneath” the second feature may mean that the first feature is directly below or obliquely below the second feature, or simply means that the first feature has a lower level than the second feature. It should be noted that when an element is referred to as being “fixed to” or “disposed on” another element, it can be directly on the other element or intervening elements may also be present. When an element is referred to as being “connected to” another element, it can be directly connected to the other element or intervening elements may also be present. The terms “vertical”, “horizontal”, “upper”, “lower”, “left”, “right” and the like used herein are for illustrative purposes only and do not imply that they are the only means of implementation.

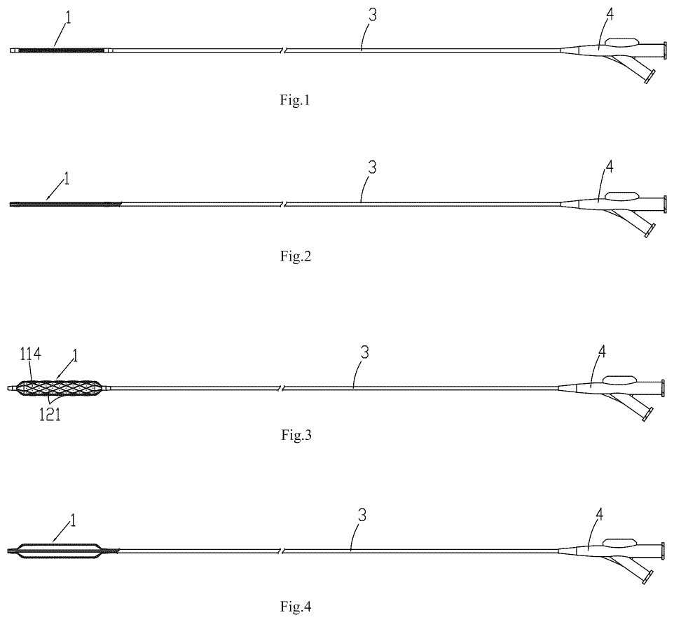

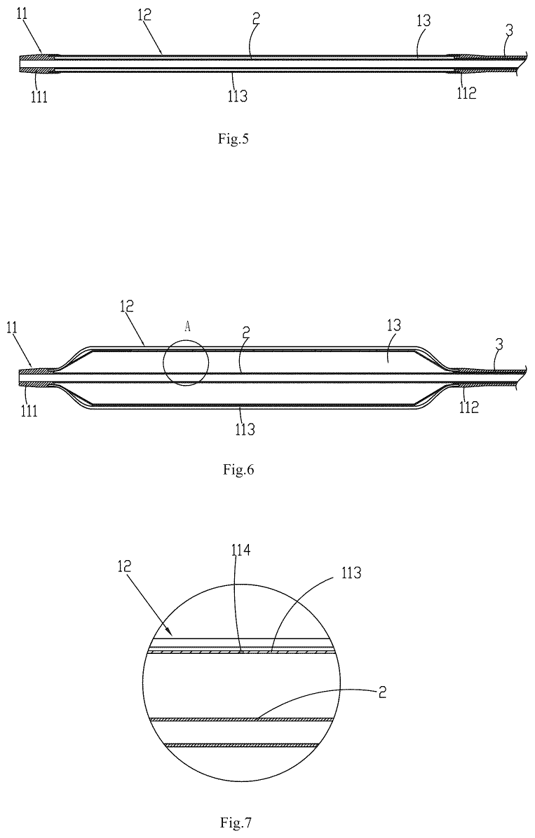

FIGS. 1 to 6 show an exemplary vascular stenosis treatment apparatus, which includes a treatment assembly 1, a first catheter 2, a second catheter 3, and a catheter seat 4.

The treatment assembly 1 includes a balloon 11 and a covering member 12, and the covering member 12 covers the outside of the balloon 11 (see FIG. 5 and FIG. 6). The balloon 11 has an unexpanded state and an expanded state, wherein the outer diameter of the balloon 11 when it is in unexpanded state is less than that of the balloon 11 when it is in expanded state; the covering member 12 expands with the expansion of the balloon 11. The covering member 12 is driven to expand together through the expansion of the balloon 11, since the overall stiffness of the covering member 12 is greater than that of the balloon 11, it can improve the expansion effect of the treatment assembly 1 on the vascular stenosis site, prevent the balloon 11 from being squeezed and deformed by the stenosis site due to insufficient expansion tension, thereby inhibiting the occurrence rate of vascular restenosis, especially suitable for the treatment of atherosclerotic stenosis and severe calcified stenosis.

The distal end of the balloon 11 is fixedly connected to or integrally formed with the distal end of the first catheter 2 to seal the distal end of the balloon 11 and the distal end of the first catheter 2. The proximal end of the balloon 11 is fixedly connected to or integrally formed with the distal end of the second catheter 3 to seal the proximal end of the balloon 11 and the distal end of the second catheter 3. The second catheter 3 is sleeved outside the first catheter 2, a drug delivery channel 13 is formed between the first catheter 2 and the second catheter 3, and the drug delivery channel 13 is in communication with the balloon 11 so that the drug can be delivered to the inside of the balloon 11 through the drug delivery channel 13 to expand the balloon 11.

As shown in FIG. 5 and FIG. 6, in some implementations, the balloon 11 includes a first head portion 111 at the distal end, a second head portion 112 at the proximal end, and a balloon body 113 fixedly connected to the first head portion 111 and the second head portion 112. The interior of the first head portion 111 and the interior of the second head portion 112 are respectively formed with channels that extend along the axial direction, and the inner diameter of the channel formed in the second head portion 112 is larger than that of the channel formed in the first head portion 111. The distal end of the first catheter 2 is fixedly connected to or integrally formed with the proximal end of the first head portion 111, the interior of the first catheter 2 is formed with a cavity channel that extends along the axial direction, the inner diameter of the cavity channel of the first catheter 2 is equal to the inner diameter of the channel formed in the first head portion 111, and the inner wall of the first catheter 2 is flush with the inner wall of the first head portion 111, so that a guide wire passes through from the cavity channel of the first catheter 2 and the channel within the first head portion 111. The distal end of the second catheter 3 is fixedly connected to or integrally formed with the proximal end of the second head portion 112, the interior of the second catheter 3 is formed with a cavity channel that extends along the axial direction, the inner diameter of the cavity channel of the second catheter 3 is equal to the inner diameter of the channel formed in the second head portion 112, and the inner wall of the second catheter 3 is flush with the inner wall of the second head portion 112, so that the drug solution flows within the drug delivery channel 13.

The distal end of the covering member 12 is fixedly connected to the first head portion 111, the proximal end of the covering member 12 is fixedly connected to the second head portion 112, the covering member 12 extends on the peripheral surface of the entire balloon 11 (see FIG. 5 and FIG. 6), so that during the expansion of the covering member 12 along with the balloon 11, the covering member 12 is not easily separated from the balloon 11, thereby reducing the risk of vascular wall injury caused by the covering element 12 falling off and separating into other locations of the blood vessel due to its detachment from the balloon 11.

In some implementation methods, the covering member 12 cannot self-expand and can only expand under the expansion force of the balloon 11. In some embodiments, it may be advantageous to provide the covering member 12 to expand synchronously with the balloon 11, that is, in the expansion state and/or during the process of moving from the unexpanded state towards the expanded state, the balloon 11 is always attached to the covering member 12; of course, the definition that balloon 11 is attached to the covering member 12 does not limit the full area of contact between the balloon 11 and the covering member 12, as long as the parts where the balloon 11 generates a force on the covering member 12 are in contact, it can be considered that balloon 11 is attached to the covering member 12. By expanding the covering member 12 along with the balloon 11, it can be ensured that the relative position between the covering member 12 and the balloon 11 remains basically unchanged, thereby facilitating the correspondence between first holes 114 of the balloon 11 and second holes 124 of the covering member 12 (as detailed below).

In some embodiments, the covering member 12 is provided that part or all of the covering member 12 moves relative to the outer surface of the balloon 11 to generate a cutting action when the balloon 11 is expanded, so that when the balloon 11 is expanded, the covering member 12 is expanded to cut the intravascular lesion site. In some cases, when the balloon 11 is in the expanded state or in the unexpanded state, the contact area between part or all of the covering member 12 and the peripheral surface of the balloon 11 is changed to realize the cutting of the intravascular lesion site by the covering member 12; that is to say, during the expansion process of the covering member 12, part or all of the covering member 12 can be flipped at a certain angle, which can become vertical from being closely attached to the outer surface of the balloon 11, thereby cutting the intravascular lesion site.

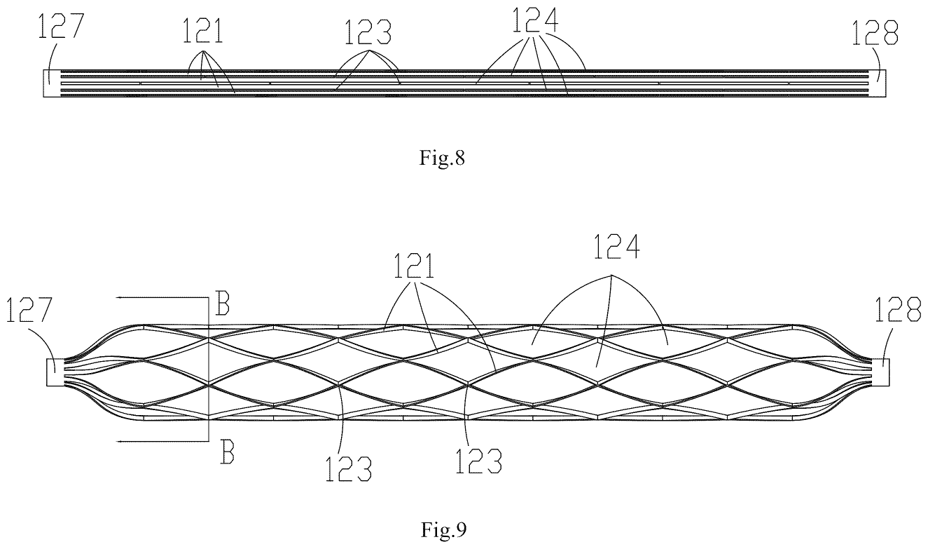

In some embodiments, a first aspect of covering member 12 is as shown in FIG. 8 and FIG. 9, the covering member 12 includes a plurality of covering elements 121, a plurality of connection points 123, a first connecting member 127, and a second connecting member 128. The covering member 12 in this aspect is formed by cutting a cylindrical tube, for example, a portion of the tube can be cut off by laser to form the plurality of second holes 124, and the remaining portion of the tube forms the plurality of covering elements 121, the plurality of connection points 123, the first connecting member 127, and the second connecting member 128, in this embodiment, the first connecting member 127 and the second connecting member 128 are circular ring-shaped. Of course, when cutting the cylindrical tube, laser can be used to cut off some of the tube at both ends, so that the first connecting member 127 and the second connecting member 128 have a closed cyclic structure having peaks and valleys arranged regularly along the circumferential direction with the axis line of the covering member 12 as the axis, compared with a circular structure, the first connecting member 127 and the second connecting member 128 of this structure can be better fixed with the balloon 11, thereby improving the firmness of the connection between the balloon 11 and the covering member 12.

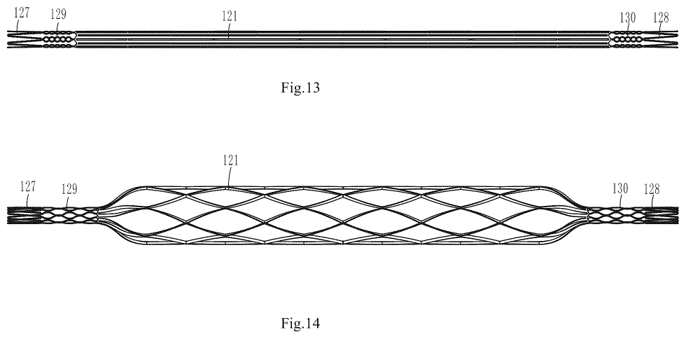

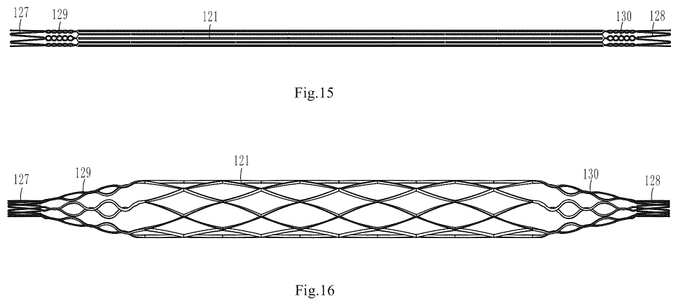

In some other embodiments, the second aspect and the third aspect of the covering member 12 are as shown in FIG. 13 to FIG. 16, the covering member 12 includes a plurality of covering elements 121, a plurality of connection points 123, a first connecting member 127 located at the distal end of the covering member 12, a second connecting member 128 located at the proximal end of the covering member 12, a first extension member 129 fixedly connected to the proximal end of the first connecting member 127 and the distal ends of the covering elements 121 and capable of be elongated or shortened, and a second extension member 130 fixedly connected to the distal end of the second connecting member 128 and the proximal ends of the covering elements 121 and capable of be elongated or shortened. The first connecting member 127 is fixedly connected to the distal end of the balloon 11, and the second connecting member 128 is fixedly connected to the proximal end of the balloon 11; the length of the first extension member 129 when the balloon 11 is in the unexpanded state is less than the length of the first extension member 129 when the balloon 11 is in the expanded state, and the length of the second extension member 130 when the balloon 11 is in the unexpanded state is less than the length of the second extension member 130 when the balloon 11 is in the expanded state. This implementation, by adding the first extension member 129 and the second extension member 130 that can elongate with the expansion of the balloon 11, can better compensate for the length reduction caused by the expansion of the covering elements 121, which is more conducive to use with large-sized balloons. The first connecting member 127 and the second connecting member 128 of this implementation are the same as the previous implementation, and will not be repeated here. The first extension member 129 and the second extension member 130 can be any structure that can achieve elongation or shortening, and the first extension member 129 and the second extension member 130 shown in FIGS. 13 to 16 are wavy and have a plurality of wavy lines, and in the unexpanded state, the plurality of wavy lines is uniformly distributed along the circumferential direction with the axis line of the covering member 12 as the axis. Specifically, one end of a wavy line is fixedly connected to end portions of two adjacent covering elements 121, and each wavy line is fixedly connected to a connecting member at a corresponding end.

Wherein, FIG. 13 and FIG. 14 are schematic diagrams of the second aspect, in this embodiment, the positions where the diameters of the two ends of the balloon 11 remain unchanged are relatively long, so that the first connecting member 127, the first extension member 129, the second extension member 130, and the second connecting member 128 are all located in the positions where the diameter of the balloon 11 remain unchanged, when transitioning from the unexpanded state to the expanded state, the expansion of the balloon 11 causes the covering member 12 to expand, the first extension member 129 and the second extension member 130 elongate until in the expanded state, the connection between the first extension member 129 and the covering elements 121, as well as the connection between the second extension member 130 and the covering elements 121, are located in the positions where the diameter of the balloon 11 becomes to increase when it transitions from the unexpanded state to the expanded state.

Wherein, FIG. 15 and FIG. 16 are schematic diagrams of the third aspect, in this embodiment, the positions where the diameters of the two ends of the balloon 11 remain unchanged are similar to the first implementation, so that the first connecting member 127 and the second connecting member 128 are located in the positions where the diameter of the balloon 11 remain unchanged, the connection between the first connecting member 127 and the first extension member 129, as well as the connection between the second connecting member 128 and the second extension member 130, are located in the positions where the diameter of the balloon 11 becomes to increase when it transitions from the unexpanded state to the expanded state, when transitioning from the unexpanded state to the expanded state, the expansion of the balloon 11 causes the covering member 12 to expand, and the diameter of the end portions close to the covering elements 121 increases at the same time when the first extension member 129 and the second extension member 130 elongate.

The material of the covering member 12 is metal, including but not limited to the group consisting of memory alloy, stainless steel, titanium alloy, nickel alloy, cobalt-chromium alloy, and combinations thereof.

Wherein, as shown in FIG. 5 and FIG. 6, the first connecting member 127 is fixedly connected to the first head portion 111 of the balloon 11 and the distal ends of the covering elements 121, respectively, and the second connecting member 128 is fixedly connected to the second head portion 112 of the balloon 11 and the proximal ends of the covering elements 121, respectively, and the covering elements 121 extend on the peripheral surface of the entire balloon 11.

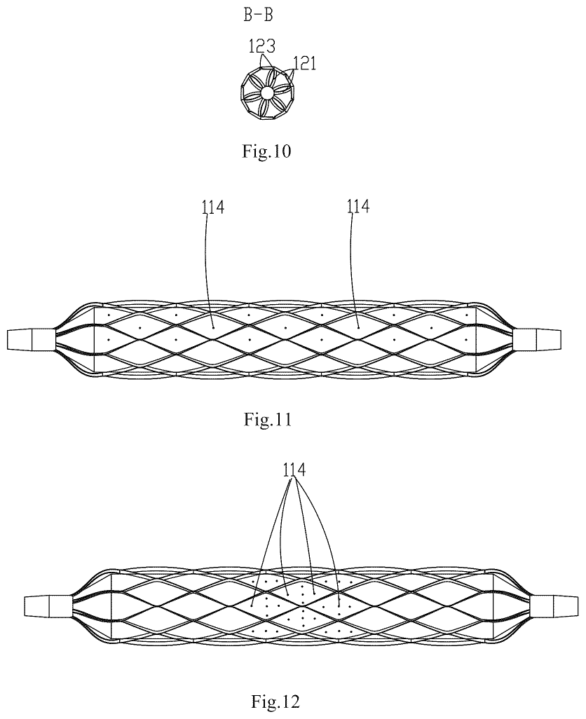

In this embodiment, as shown in FIG. 8, when the covering member 12 is in the unexpanded state, each of the covering elements 121 has a length extending along the axial direction of the covering member 12, and the plurality of covering elements 121 is uniformly distributed along the circumferential direction of the covering member 12. According to some implementations, the number of covering elements 121 is 10˜14, as shown in FIG. 10, and the number of covering elements 121 is 12. In the unexpanded state of the covering element 12, the width of the covering elements 121 is greater than the thickness of the covering elements 121, as the balloon 11 expands, the covering elements 121 are partially or completely flipped, causing the covering elements 121 in contact with the intravascular stenosis site to form an elongated sheet shape, and the surface of the covering element 121 in contact with the intravascular stenosis site is relatively thin, which has better cutting effect, moreover, in the expanded state, a drug delivery space with a certain distance is formed between the balloon 11 and the vessel wall, which is conducive to the action of the drug on the vessel wall (see FIG. 9). Further, the width of the covering elements 121 is 2˜10 times the thickness of the covering elements 121, and more further, the thickness of the covering elements 121 may be 0.02˜0.2 mm.

As shown in FIG. 8 and FIG. 9, two adjacent covering elements 121 are fixedly connected through the plurality of connection points 123, and two adjacent connection points 123 on the same covering element 121 are respectively located on two opposite sides of the covering element 121 and are staggered with each other. Further, the plurality of connection points 123 are respectively located in a plurality of planes parallel to each other, the planes are perpendicular to the axis line of the covering member 12, and the distances between two adjacent planes are equal, so that the shapes of the second holes 124 are roughly the same. Further, the connection points 123 have a length of 0.5˜3 mm; the connection points 123 have a thickness of 0.02˜0.2 mm; the connection points 123 have a width of 0.1˜0.5 mm. More further, the thickness of the connection points 123 may be equal to the thickness of the covering elements 121.

In this disclosure, the thickness of a component refers to the distance between two surfaces of the component in the radial direction of the treatment apparatus, the width of the component refers to the distance between two sides of the component in the circumferential direction of the treatment apparatus, the length of the component refers to the distance between two end faces of the component in the axial direction of the treatment apparatus, and the thickness, width, and length are all measured values when the treatment apparatus is in the unexpanded state.

As shown in FIG. 8, FIG. 9, FIGS. 13 to 16, the plurality of covering elements 121 and the plurality of connection points 123 together form a plurality of second holes 124, and in the expanded state, the cross-sectional area of the second holes is 10˜50 mm². When the covering member 12 transitions from the unexpanded state to the expanded state, the covering elements 121 are flipped, that is, the faces of the covering elements 121 that originally attached to the balloon 11 will move away from the balloon 11, so that the covering elements 121 can cut the vascular lesion site while expanding, and due to the narrow thickness of the covering elements 121, it can have a better cutting effect on the vascular lesion site. When the covering elements 121 are flipped, two adjacent connection points 123 connected to the same covering element 121 move respectively towards directions close to the balloon 11 and away from the balloon 11. When the covering member 12 is in the expanded state, the second holes 124 are roughly rhombic, and in the same rhombus, the connection points 123 at two opposite vertexes of this rhombus are located on the inner side of the covering member 12, and the connection points 123 at the other two opposite vertexes of this rhombus are located on the outer side of the covering member 12, and the covering elements 121 enclose outside the first holes 114 to form a plurality of independent drug delivery spaces.

The design of the structure of the covering member 12 ensures that the covering member 12 expands along with the balloon 11 and has good support, providing positioning for the balloon 11 and allowing the drug solution to accurately act on the desired area, improving the accuracy of treatment.

In order to avoid drug loss during the delivery process of the drug loaded balloon 11, this disclosure provides a plurality of first holes 114 on the wall of the balloon 11 to achieve the expansion of the balloon 11 and the spray of the drug by delivering the therapeutic drug solution into the balloon 11, reducing drug loss during delivery, increasing drug concentration, and making the dosage more precise and controllable.

By controlling the number and aperture of the first holes 114, the resistance of the drug solution to seep out from inside the balloon 11 can be controlled, thereby ensuring that the drug is sprayed out of the balloon 11 only under a set pressure. Due to the insertion of balloon 11 into the blood vessel, the blood and other substances in the vessel exert a certain pressure on balloon 11, therefore, during the expansion of balloon 11, the external pressure of the balloon 11 can prevent the drug solution inside the balloon 11 from flowing out of the first holes 114 or allow only a small amount to seep out from the first holes 114, thereby reducing the loss of the drug solution, and it can achieved that the drug solution can be sprayed out from the first holes 114 in the expanded state, and the drug solution remains inside the balloon 11 or only a small amount of drug solution seeps out from the first holes 114 in the unexpanded state and during the process of moving from the unexpanded state towards the expanded state. Further, in the expanded state, the aperture of the first holes 114 is 20˜70 μm. Wherein, spraying refers to that the drug solution can be ejected in a linear shape, while seeping refers to that the drug solution only has liquid overflow on the surface of the balloon 11, in the form of water droplets.

Wherein, by controlling the size, number, and distribution of the first holes 114, it is possible to control the spraying of the drug solution from the first holes 114 while or after the cutting of the covering member 12. The first holes 114 can be distributed on the entire peripheral surface of balloon 11 as shown in FIG. 3, or it can be only opened on the upper half of the balloon 11 as shown in FIG. 11. The distribution position of the first holes 114 can be adjusted according to the location of the intravascular lesion site that needs to be administered.

In the expanded state, the aperture of the first holes 114 is less than that of the second holes 124, the covering elements 121 enclose outside the first holes 114, and the position of the plurality of first holes 114 corresponds to the position of the plurality of second holes 124, such that a plurality of relatively independent drug delivery spaces are formed between the balloon 11 and the covering member 12. Wherein, one second hole 124 is provided corresponding to one or more first holes 114. For example, FIG. 3 and FIG. 11 show that one second hole 124 corresponds to one first hole 114, in which the axis line of the first hole 114 coincides with the axis line of the second hole 124. For another example, one of the second holes 124 corresponding to two or more of the first holes 114 is shown in FIG. 12, further, one of the second holes 124 corresponds to 2˜5 of the first holes 114. In order to ensure the speed of administration and the uniformity of the drug solution, two or more first holes 114 may be uniformly distributed within the range of the second hole 124.

In some embodiments, in the unexpanded state, the position of the first holes 114 and the position of the second holes 124 also correspond, which is conducive to ensuring that the position of the first holes 114 and the position of the second holes 124 correspond after expansion, and it is not easy for the first holes 114 to be covered by the covering elements 121.

The coverage rate of the peripheral surface of the balloon 11 by the covering member 12 in the unexpanded state is greater than that of the peripheral surface of the balloon 11 by the covering member 12 in the expanded state, and in the unexpanded state, the second holes 124 are in the shape of an elongated narrow slit. When the balloon 11 transitions from the unexpanded state to the expanded state, both the first holes 114 on the balloon 11 and the second holes 124 on the covering member 12 undergo deformation, and when the balloon 11 is in the expanded state, the degree of deformation of the second holes 124 is greater than that of the first holes 114, and when transitioning from the unexpanded states to the expanded state, both the first holes 114 and the second holes 124 become larger.

Referring to FIG. 1 to FIG. 4, the proximal portion of the first catheter 2 and the proximal portion of the second catheter 3 are respectively connected to the catheter seat 4, and their connection mode can be arbitrarily combined, as long as they can achieve the expansion of the balloon 11 and the infusion of the drug solution into the blood vessels. For example, their connection mode may be that the first catheter 2 is slidably connected to the catheter seat 4, and the second catheter 3 is fixedly connected to the catheter seat 4, or that the first catheter 2 is fixedly connected to the catheter seat 4, and the second catheter 3 is slidably connected to the catheter seat 4, or that both the first catheter 2 and the second catheter 3 are slidably connected to the catheter seat 4, so that the axial displacement of the balloon 11 in the unexpanded state and the expanded state can be achieved through the relative sliding between the catheters and the catheter seat 4. Of course, their connection mode may also be that the proximal portion of the first catheter 2 and the proximal portion of the second catheter 3 are fixedly connected to the catheter seat 4, respectively.

The usage method of the treatment apparatus in this application is carried out according to the usual usage method of intervention catheters, the apparatus is placed in place, and the balloon 11 is pressurized and opened from the catheter seat 4, the covering member 12 is expanded together with the balloon 11 under the force applied by the balloon 11, the covering member 12 forms a cut on the intravascular stenosis site during the expansion process, and then pressurization continues to a certain pressure, and when the pressure is higher than a set value, the drug solution continues to spray out, at the same time, the drug solution is restricted within the limited drug delivery spaces enclosed by the vessel wall, the covering elements 121 and the wall of the balloon 11, achieving the goal of uniform administration of the drug to the vessel wall.

The above detailed describes the present disclosure, and is intended to make a person familiar with the technology being able to understand the content of the present disclosure and thereby implement it, and should not limit the protective scope of this disclosure. Any equivalent variations or modifications according to the spirit of the present disclosure should be covered by the protective scope of the present disclosure.

1. A vascular stenosis treatment apparatus, comprising:
a treatment assembly having:
a balloon, the wall of the balloon being provided with a plurality of first holes for liquid to pass through, and the balloon having an unexpanded state and an expanded state; and
a covering member, the covering member covering the outside of the balloon and expanding with the expansion of the balloon, the covering member having a plurality of second holes, and when the balloon is in the expanded state, the aperture of the first holes being smaller than the aperture of the second holes.

2. The vascular stenosis treatment apparatus according to claim 1, wherein, when the balloon transitions from the unexpanded state to the expanded state, both the first holes on the balloon and the second holes on the covering member become larger, and when the balloon is in the expanded state, the degree of enlargement of the second holes is greater than that of the first holes.

3. The vascular stenosis treatment apparatus according to claim 1, wherein, the coverage rate of the peripheral surface of the balloon by the covering member in the unexpanded state is greater than that of the peripheral surface of the balloon by the covering member in the expanded state; and/or,
in the expanded state and/or during the process of moving from the unexpanded state to the expanded state, the balloon and the covering member are attached to each other.

4. The vascular stenosis treatment apparatus according to claim 1, wherein, in the expanded state, the position of the plurality of first holes corresponds to the position of the plurality of second holes, such that a plurality of relatively independent drug delivery spaces are formed between the balloon and the covering member; and/or, in the unexpanded state, the position of the first holes corresponds to the position of the second holes; and/or, one second hole is provided corresponding to one or more first holes.

5. The vascular stenosis treatment apparatus according to claim 4, wherein, when one second hole is provided corresponding to one first hole, the axis line of the first hole coincides with the axis line of the second hole; when one second hole is provided corresponding to two or more first holes, the two or more first holes are uniformly distributed within the range of the second hole; and/or
one second hole is provided corresponding to 2˜5 first holes.

6. The vascular stenosis treatment apparatus according to claim 1, wherein, the number and aperture of the first holes are provided so that, in the expanded state, the drug solution can be sprayed out from the first holes, and in the unexpanded state and during the process of moving from the unexpanded state to the expanded state, the drug solution remains inside the balloon or only a small amount of drug solution seeps out from the first holes; and/or
in the expanded state, the cross-sectional area of the second holes is 10˜50 mm², and/or, the shape of the second holes is rhombus, and/or, the aperture of the first holes is 10 μm˜80 μm.

7. The vascular stenosis treatment apparatus according to claim 1, wherein, the distal end of the covering member is fixedly connected to the distal end of the balloon, and the proximal end of the covering member is fixedly connected to the proximal end of the balloon; and/or
the vascular stenosis treatment apparatus further comprises a first catheter partially located within the balloon and connected to the distal end of the balloon in a sealed manner or integrally formed with the distal end of the balloon, a second catheter sleeved outside the first catheter and connected to the proximal end of the balloon in a sealed manner or integrally formed with the proximal end of the balloon, and a catheter seat respectively connected to the proximal end of the first catheter and the proximal end of the second catheter, forming a drug delivery channel between the first catheter and the second catheter.

8. The vascular stenosis treatment apparatus according to claim 1, wherein, the covering member is provided that part or all of the covering member moves relative to the outer surface of the balloon to generate a cutting action when the balloon is expanded.

9. The vascular stenosis treatment apparatus according to claim 8, wherein, when the balloon transitions from the unexpanded state to the expanded state, the contact area between part or all of the covering member and the peripheral surface of the balloon is changed to realize the cutting of an intravascular lesion site by the covering member; and/or
during or after the covering member carries out the cutting, the drug solution is sprayed out from the first holes.

10. The vascular stenosis treatment apparatus according to claim 9, wherein, the covering member comprises a plurality of covering elements and a plurality of connection points;
when the covering member is in the unexpanded state, each of the covering elements has a length extending along the axial direction of the covering member, and the plurality of covering elements is distributed along the circumferential direction;
two adjacent covering elements are fixedly connected through the plurality of connection points, and two adjacent connection points on the same covering element are respectively located on two opposite sides of the covering element and are staggered with each other;
when the covering member transitions from the unexpanded state to the expanded state, the covering elements are flipped, and two adjacent connection points connected to the same covering element move respectively towards directions close to the balloon and away from the balloon.

11. The vascular stenosis treatment apparatus according to claim 10, wherein, the plurality of connection points is respectively located in a plurality of planes parallel to each other, the planes are perpendicular to the axis line of the covering member, and the distances between two adjacent planes are equal; and/or
when the covering member is in the unexpanded state, the width of the covering elements is greater than the thickness of the covering elements; and/or
the connection points have a length of 0.5˜3 mm; and/or, the connection points have a thickness of 0.02˜0.2 mm; and/or, the connection points have a width of 0.1˜0.5 mm; and/or, the thickness of the connection points is equal to the thickness of the covering elements; and/or,
the plurality of covering elements and the plurality of connection points together form the plurality of second holes, and in the expanded state, the covering elements enclose outside the first holes to form a plurality of independent drug delivery spaces; and/or,
when the covering member is in the expanded state, the second holes are rhombic, and in the same rhombus, the connection points at two opposite vertexes of this rhombus are located on the inner side of the covering member, and the connection points at the other two opposite vertexes of this rhombus are located on the outer side of the covering member.

12. The vascular stenosis treatment apparatus according to claim 11, wherein, when the covering member is in the unexpanded state, the width of the covering elements is 2˜10 times the thickness of the covering elements.

13. The vascular stenosis treatment apparatus according to claim 8, wherein, the covering member is formed by cutting a cylindrical tube; and/or, the material of the covering member is metal; and/or, the material of the covering member is selected from the group consisting of memory alloy, stainless steel, titanium alloy, nickel alloy, cobalt-chromium alloy, and combinations thereof.

14. The vascular stenosis treatment apparatus according to claim 10, wherein, the covering member further comprises a first connecting member located at the distal end of the covering member and a second connecting member located at the proximal end of the covering member, the first connecting member is fixedly connected to the distal end of the balloon and the distal ends of the covering elements, respectively, and the second connecting member is fixedly connected to the proximal end of the balloon and the proximal ends of the covering elements, respectively; and/or
the covering member further comprises a first connecting member located at the distal end of the covering member, a second connecting member located at the proximal end of the covering member, a first extension member fixedly connected to the proximal end of the first connecting member and the distal ends of the covering elements and capable of being elongated or shortened, and a second extension member fixedly connected to the distal end of the second connecting member and the proximal ends of the covering elements and capable of being elongated or shortened, the first connecting member is fixedly connected to the distal end of the balloon, and the second connecting member is fixedly connected to the proximal end of the balloon; the length of the first extension member when the balloon is in the unexpanded state is less than that of the first extension member when the balloon is in the expanded state, and the length of the second extension member when the balloon is in the unexpanded state is less than that of the second extension member when the balloon is in the expanded state.

15. The vascular stenosis treatment apparatus according to claim 14, wherein, the balloon comprises a first head portion at the distal end, a second head portion at the proximal end, and a balloon body fixedly connected to the first head portion and the second head portion, the first connecting member is fixedly connected to the first head portion, the second connecting member is fixedly connected to the second head portion, and the covering elements extend on the peripheral surface of the entire balloon; and/or,
the first connecting member and the second connecting member are respectively circular rings, or are respectively closed rings having peaks and valleys regularly arranged along the circumferential direction with the axis line of the covering member as the axis; and/or,
in the unexpanded state, the first extension member and the second extension member are respectively a plurality of wavy lines uniformly distributed along the circumferential direction with the axis line of the covering member as the axis; and/or,
in the expanded state, the connections between the first extension member and the covering elements and the connections between the second extension member and the covering elements are located in the positions where the diameter of the balloon becomes to increase when it transitions from the unexpanded state to the expanded state; and/or,
the connection between the first connecting member and the first extension member and the connection between the second connecting member and the second extension member are located in the positions where the diameter of the balloon becomes to increase when it transitions from the unexpanded state to the expanded state.

16. The vascular stenosis treatment apparatus according to claim 2, wherein, the coverage rate of the peripheral surface of the balloon by the covering member in the unexpanded state is greater than that of the peripheral surface of the balloon by the covering member in the expanded state; and/or,
in the expanded state and/or during the process of moving from the unexpanded state to the expanded state, the balloon and the covering member are attached to each other.

17. The vascular stenosis treatment apparatus according to claim 7, wherein, the covering member is provided that part or all of the covering member moves relative to the outer surface of the balloon to generate a cutting action when the balloon is expanded.
